(19) United States
(12) Patent Application Publication
(10) Pub. No.: US 2018/0272042 A1
(43) Pub. Date: Sep. 27, 2018

(54) DRUG-POLYMER FILM FOR CONTROLLED LOCAL DELIVERY AT TISSUE-DEVICE INTERFACE

(71) Applicant: Brown University, Providence, RI (US)

(72) Inventors: David Borton, Providence, RI (US); Elaina Atherton, Providence, RI (US); Vicki Colvin, Providence, RI (US)

(21) Appl. No.: 15/933,293

(22) Filed: Mar. 22, 2018

Related U.S. Application Data
(60) Provisional application No. 62/474,763, filed on Mar. 22, 2017.

(51) Int. Cl. A61L 31/06; A61L 31/16; A61L 31/02; A61L 31/04; A61K 31/05; A61B 5/04; A61B 17/68; A61C 8/00; A61F 2/00; A61F 2/14; A61N 1/375

(52) U.S. Cl. CPC A61L 31/06; A61L 31/16; A61L 31/026; A61L 31/048; A61K 31/05; A61B 5/04001; A61F 2002/183; A61C 8/0013; A61F 2/0077; A61F 2/14; A61N 1/37512; A61L 2420/08; A61B 17/68

(57) ABSTRACT

A polymer film coating for medical devices and medical devices with coating are provided to controllably release one or more therapeutics or other agents locally at the tissue-device interface for a prolonged period of time. The coating can be prepared via layer by layer (LbL) assembly of polymers and/or polymers conjugated to or otherwise associated with one or more drugs, particularly recyclable antioxidants or antioxidant regulators. The resulting coating contains alternating layers that are assembled via electrostatic interactions, hydrogen bonding, or other secondary interactions. The coated device can provide for long term local delivery of antioxidants, preferably antioxidant upregulators and/or recyclable antioxidants, to modulate the oxidative stress associated with brain implants.

### CROSS-REFERENCE TO RELATED APPLICATION

This application claims priority to U.S. Provisional Application No. 62/474,763, filed Mar. 22, 2017, the disclosure of which is incorporated by reference herein.

### FIELD OF THE INVENTION

The field of the invention generally relates to drug delivery and implantable devices.

### BACKGROUND OF THE INVENTION

Cortical recording devices have shown promises in helping people with paralysis regain function through neural control of prosthetics (Hochberg et al., “Reach and grasp by people with tetraplegia using a neurally controlled robotic arm”, Nature, 485(7398), 372-375 (2012); Hochberg et al., “Neuronal ensemble control of prosthetic devices by a human with tetraplegia”, Nature, 442(7099), 164-171 (2006); Wang et al., “An Electrocorticographic Brain Interface in an Individual with Tetraplegia”, PLoS ONE, 8(2), 1-82013 (2013); Bouton et al., “Restoring cortical control of functional movement in a human with quadriplegia”, Nature, 533(7602) (2016)). These neuroprostheses sense voltage changes in the medium related to neural information exchange via direct electrode contact with neural tissue (e.g., neurons, glia, and dura), or the “neural interface”. Neural signals are then extracted via active circuitry for amplification, digitization, and interpretation (decoding) into control signals that drive an effector. While neuroprosthetics have delivered impressive demonstrations in technical feasibility, they have yet to become a therapy for individuals with a damaged or diseased nervous system (Collinger et al., “Neuroprosthetic technology for individuals with spinal cord injury”, The Journal of Spinal Cord Medicine, 36(4), 258-272 (2013); Lu, et al., “Current Challenges to the Clinical Translation of Brain Machine Interface Technology”. 1st edn, International Review of Neurobiology (Elsevier Inc. 2012). A problem with many existing and commercially available neuroprosthetics has been the limited ability to record neural activity with consistency over months to years.

Current devices and methods to record neural activity greatly rely on physical proximity of probes or sensors to the local electric fields developed by neurons to achieve high spatial and temporal resolution. Such proximity often requires probe implantation, which may result in disruption of blood flow, extracellular matrix, and other cellular processes. Many studies have attributed the inconsistent signal quality of cortical recording devices to inflammatory responses and local neurodegeneration at the implant interface (Barrese, et al., “Failure mode analysis of silicon-based intracortical microelectrode arrays in non-human primates”, J. Neural Eng., 10(6), 66014 (2013); Prasad, et al., “Comprehensive characterization and failure modes of tungsten microwire arrays in chronic neural implants”, J. Neural Eng., 9(5), 56015 (2012); McConnell, et al., “Implanted neural electrodes cause chronic, local inflammation that is correlated with local neurodegeneration,” J. Neural Eng., 6(5), 56003 (2009); Biran, et al., “Neuronal cell loss accompanies the brain tissue response to chronically implanted silicon microelectrode arrays”, Experimental Neurology, 195, 115-126 (2005)). After electrode implantation, where there is significant local disruption of the blood brain barrier, an influx of macrophages initiates an acute neuroinflammatory response by activating and recruiting microglia and astrocytes, which in turn produce fibrotic scar tissue to encapsulate the foreign body (so called ‘glial scar’) (see, e.g., Potter, et al., “The effect of resveratrol on neurodegeneration and blood brain barrier stability surrounding intracortical microelectrodes”, Biomaterials, 34(29), 7001-7015 (2013)). While the glial encapsulation scar stabilizes at around 6 weeks, the signal quality of the electrode recordings continues to deteriorate (e.g., loss of signal, or increase in noise) and often fails entirely in about a year, a phenomenon which has been correlated with the timing of neural cell death around the electrode (see Xie et al., “In vivo monitoring of glial scar proliferation on chronically implanted neural electrodes by fiber optical coherence tomography”, Frontiers in Neuroengineering, 7(Aug.), 34 (2014); McConnell et al., “Implanted neural electrodes cause chronic, local inflammation that is correlated with local neurodegeneration. Journal of Neural Engineering, 6(5), 56003 (2009); Potter et al., (2013)).

Recent studies have indicated that even the glial scar alone is unlikely to produce the deleterious effects seen on neural recordability over time (Malaga et al. 2016). It has been hypothesized that many of the adverse responses to cortical array implantations may be due to oxidative stress (OS), a process in which reactive oxygen species (ROS) is overproduced as part of the inflammatory signalling cascade in a “frustrated phagocytosis” response (Potter-Baker, K. A., & Capadona, J. R., “Reducing the “Stress”: Antioxidative Therapeutic and Material Approaches May Prevent Intracortical Microelectrode Failure”, ACS Macro Letters, 4(3), 275-279 (2015); Polikov, et al., “Response of brain tissue to chronically implanted neural electrodes”, J. Neuroscience Methods, 148(1), 1-18 (2005); Biran, 2005; Potter et al., “Stab injury and device implantation within the brain results in inversely multiphasic neuroinflammatory and neurodegenerative responses. J. Neural Eng., 9(4), 46020 (2012)). In this response, glia produce many inflammatory molecules, including ROS (Block, et al, “Microglia-mediated neurotoxicity: uncovering the molecular mechanisms. Nature Reviews, 8, 57-69 (2007)). The ROS is initially successful at activating additional microglia, and reacting with endothelial tight junctions to recruit more inflammatory molecules (Schreibelt et al., “Reactive oxygen species alter brain endothelial tight junction dynamics via RhoA, PI3 kinase, and PKB signaling”, The FASEB Journal, 21(13), 3666-3676 (2007)). During this time, neurons are able to produce enough endogenous antioxidants to neutralize any interactions with excess ROS. However, as the ROS production continues to increase due to the chronic presence of a foreign body, the oxidative load outweighs the antioxidant production ability of neurons, resulting in lipid, protein, and DNA peroxidation (Block, et al, 2007; Mittal et al., “Reactive oxygen species in inflammation and tissue injury”, Antioxidants & Redox Signaling, 20(7), 1126-1167 (2014)), as well as cellular cascades to trigger programmed cell death (Chang et al., “Effect of hyperoxia on cortical neuronal nuclear function and programmed cell death mechanisms”, Neurochemical Research, 32(7), 1142-1149 (2007)).

Similar problems are observed with other implantable devices and biomaterials. For example blood/material interactions occur with a variety of implantable biomaterials and devices such as hemodialyzers, oxygenators, catheters, prostheses, stents, vascular grafts, and other devices and materials following implantation.

Therefore, it is an object of the present invention to provide a coating for improving implantable devices.

It is also an object to provide a coating for improving implantable devices that contain an electrode, such as a neural activity sensor or modulator.

It is also an object of the present invention to provide improved implantable devices, optionally with increased longevity following implantation.

It is also an object of the present invention to provide a method to make and use such coatings and devices, optionally to provide a method to increase the useful life for such devices following implantation.

### SUMMARY OF THE INVENTION

An implantable device coated with a multilayered polymer film containing an active agent incorporated therein is provided. Also provided are coatings for implantable devices and methods for making and using such coatings.

The coating is generally a multilayer film, which contains at least a first layer and a second layer adjacent to the first layer wherein the first layer comprises a first polymeric material and at least first moiety wherein the second layer comprises a second polymeric material and at least second moiety, where the charge on the second moiety is opposite the charge on the first moiety or wherein the first and second moieties otherwise have affinity for each other. The first and second moieties on the adjacent layers interact with one another so that the adjacent layers associate with each other forming a bilayer (e.g. via non-covalent interactions, such as via electrostatic interactions or hydrogen bonding). One or more bilayers are included in a film that forms a coating on a substrate or the surface of a device. Each of the polymers may be deposited on the substrate or the surface of the device in an alternating sequence to form a layer-by-layer (LbL) film.

Additionally the active agent for delivery (an antioxidant, or prodrug thereof, optionally two or more antioxidants, or prodrugs thereof, optionally with another drug) is incorporated into at least one layer or between layers in a bilayer such that decomposition of one or more layers of the coating results in release of the antioxidant or other active agent.

Optionally, one or more of the bilayers in a coating contains a charged drug-polymer conjugate and a polymer with an opposite charge. A layer-by-layer film may be formed based on electrostatic bonding between layers of one or more drug-polymer conjugates, or between layers of a drug-polymer conjugate and another polymer. Optionally, another active agent for delivery is incorporated in one or more of the bilayers. Optionally, the coating also contains one or more bilayers that do not contain an active agent for deliver. Further, one or more base layers may be deposited on the surface to allow for the formation of the bilayers to form a coating on the surface.

The devices may be suitable for implantation in the brain. Optionally, the devices provide for long-term local drug delivery (e.g., at one or more times such as three months, six months, twelve months following implantation) directly at the device-tissue interface in the brain, bypassing the blood brain barrier.

Prodrug-polymer conjugates are generally formed by covalently bonding a drug agent with a polymer. A degradable or non-degradable linker may be used between the drug and the polymer. Suitable polymers for the assembled film, including ones that drugs are attached to or incorporated in and ones lacking a drug component, are generally biocompatible, bioabsorbable, and/or biodegradable; and they support electrostatic, hydrogen bonding, hydrophobic attraction, or other non-covalent interactions. Exemplary drugs to be delivered, including via conjugation to a polymer and/or encapsulated in the film, include antioxidants, particularly antioxidants that are recyclable or that are also antioxidant upregulators, such as for example, pterostilbene, resveratrol, and cerium oxide nanoparticles.

The coating can have a thickness in the range of 10 nm to 10 μm. The coating typically has a thickness of about at least 1 μm, e.g., 1, 2, 3, 4, 5, 6, 7, 8, 9, or 10 μm thick. Optionally, the coating is thinner, such as having a thickness of at least 10 nm, e.g. 10 nm-30 nm, 10-50 nm, 50-100 nm, 100-200 nm, 100-500 nm, or 100-1000 nm. The coating typically contains a plurality of layers of prodrug-polymer conjugates and optionally polymers lacking prodrugs. With electrostatically assembled coatings, one or more bilayers are present in the coating, for example, about 10 bilayers, 20 bilayers or even 60 bilayers.

The coating may release the agents via disassembly of each outer layer over a period of at least about 14 months, 1 year, 10 months, 8 months, or 6 months following implantation. The film may withstand an in vivo environment for over 1, 2, 3, 4, 5, 6, 7, 8, 9, or even up to 10 years with less than 5%, 10%, 15%, 20%, 30%, 40%, or 50% loss of the film thickness. The coating is preferably resistant to enzymatic degradation or hydrolysis for at least a period of 6 months, 8 months, 10 months, 12 months, or 14 months, and prevents pre-mature leaching of the agents from the coating.

The coating may increase functional recording life of an implanted neural recording electrode compared electrode without the coating.

The coated device may provide long term local treatment of the tissue-device interface with antioxidants that disrupt inflammatory signalling cascade. Coated neuro-devices delivering antioxidants may provide a defense against long term oxidative damage and neurodegeneration.

### BRIEF DESCRIPTION OF THE DRAWINGS

FIG. 1 is a diagram showing an exemplary electrode coated with alternating layers of cationic polymer-prodrug conjugate (102a and 102b) and anionic polymer (104). The tip 100 of this exemplary electrode is not coated.

FIG. 2 is a schematic depicting a method for coating an exemplary electrode.

FIG. 3 is a schematic depicting an implanted device and delivery of antioxidants over time.

### DETAILED DESCRIPTION OF THE INVENTION

### I. Coatings

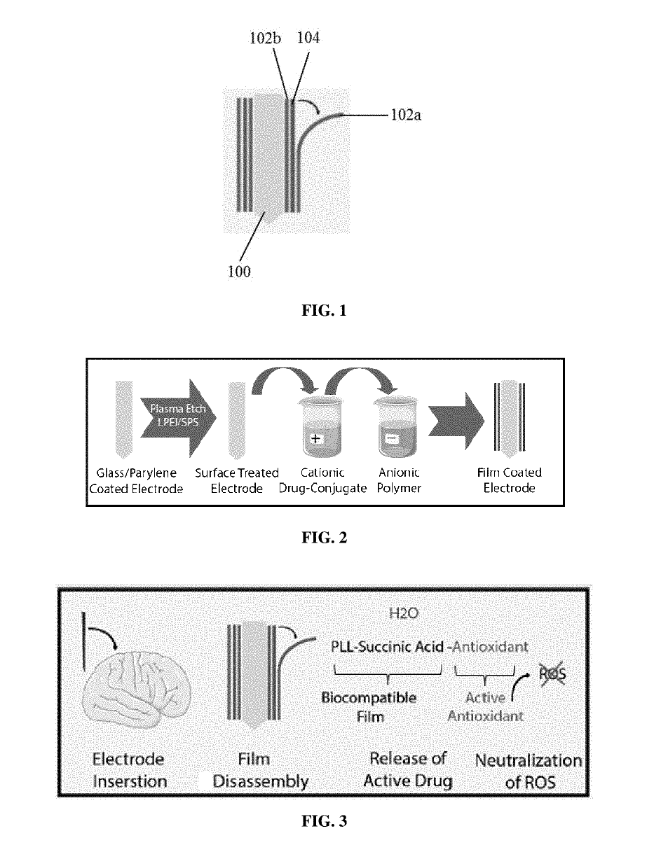

An exemplary coating on a device is depicted in FIG. 1. The coating has at least two layers that form a bilayer. Layers may be assembled via electrostatic interactions, hydrophobic interactions, hydrogen bonding interactions, or other non-covalent interactions. Optionally, the layers are assembled via electrostatic interactions between adjacent layers. For example, a charged drug-polymer conjugate and a polymer with an opposite charge may be deposited on the surface of a device in an alternating sequence to form a layer-by-layer (LbL) film. One or more base layers which generally do not contain a drug may be deposited on the surface of a device before a prodrug-polymer conjugate or the deposition of a bilayer with a drug incorporated therein.

1. Polymers

Biocompatible polymers are generally used to prepare the coating. Typically the coating is formed from two or more different polymers. For example, the coating may be formed of a plurality of bilayers, where each bilayer is formed from a first polymer containing cationic groups and a second polymer containing anionic groups.

In one embodiment, the biocompatible polymer(s) is biodegradable or bioabsorbable. In another embodiment, the polymer is non-degradable. In other embodiments, the polymers are a mixture of degradable and non-degradable polymers.

In some embodiments, the polymers are biocompatible and do not degrade by hydrolysis or enzymatic degradation for a period of at least 6 months following implantation in vivo.

Exemplary polymers for assembly of film include charged polyamino acids, poly-L-Lysine, Poly-L-Glutamic Acid, Poly-D-Lysine, poly-histidine, Linear polyethyleneimine (e.g., for base layer), branched polyethyleneimine, Sodium Poly-Styrenesulfonate (e.g., for base layer), Poly-caprolactone, Poly-ethylene oxide, and co-polymers of the above listed items. In some embodiments, one or more layers in the film contain polyacrylic acid. Optionally one or more of these polymers can be included in a copolymer that contains other polymer(s).

Poly(ethylene oxide) (PEO) may be paired with poly(acrylic acid) (PAA) in forming layer by layer assembled films. PEO and PAA interacts at low pH through hydrogen bonding between the PEO ether oxygens and the PAA acid groups. This coating may be useful for devices that are not implanted in the brain.

Copolymers of the above, such as random, block, or graft copolymers, or blends of the polymers listed above can be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

Generally, the polymers are selected to form a coating that contains the drug(s) to be delivered within its layer until that layer is at the outer surface of the implant. Thus, generally a layer will not contain natural homopolymers, such as polysaccharides (for example, chitosan, dextran, pullulan, amylopectin, mannan), or negatively charged polysaccharides (e.g., chondroitin sulfate, dextran sulfate, hyaluronic acid, heparin, pectin, alginic acid). However, these polymers may be included in one or more layers when in the form of a co-polymer, such as with one of the above-listed polymers for forming the coating. Additionally, for films in which an initial burst release is desired, the films may contain one or more of these homopolymers in its outer layers to facilitate immediate release of the drug therein.

Generally, the polymers that form that form one or more layers of the coating does not include proteins. In some forms, one or more of the layers may contain a protein, but the protein is not gelatin type A, bovine serum albumin, or fibronectin.

2. Antioxidants to be Associated with One or More Polymer Layers and/or Bilayers

The coating typically includes at least one drug which is an antioxidant. Preferably, the antioxidant is also an antioxidant upregulator or is a recyclable antioxidant. Other antioxidants may be used, as well.

Additional antioxidants that can be included in the coating include vitamin C, carotenoids, polyphenols (e.g., flavonoids such as apigenin, luteolin, tangeritin, isohametin, kaempferol, myricetin, proanthocyanidins, quercetin, eriodictyol, hesperetin, naringenin, catechin, gallocatechin, epicatechin, and derivatives thereof; phenolic acids and their esters such as chicoric acid, chlorogenic acid, cinnamic acid, ellagic acid, ellagitannins, gallic acid, gallotannins, rosmarinic acid, or salicyclic acid; and other nonflavonoid phenolics such as curcumin, flavonolignans, xanthones, or eugenol), and vitamin cofactors, and minerals. Optionally, the drug is not Vitamin E.

The antioxidant may be covalently conjugated or non-covalently associated to a polymer or bilayer forming the coating. Generally, the drug-polymer conjugate is not formed by covalently conjugating the drug to a monomer and then polymerizing the monomer to form the polymer. Rather, the drug-polymer conjugate is generally formed by covalently attaching the drug to a polymer. Optionally, a degradable or non-degradable linker may be used to attach the drug and the polymer.

Suitable antioxidant upregulators include resveratrol or an analog thereof, such as pterostilbene.

Suitable recyclable antioxidants include cerium oxide nanoparticles (CNPs), which can be coated with a hydrophilic polymer or uncoated.

The coating may include one or more antioxidants, typically in the form of a prodrug. When the drug is in the form of a prodrug, it is in an inactive form, and, after administration, it is converted within the body into a pharmacologically active drug. Including a prodrug, such as an antioxidant in the form of a prodrug, allows for greater control of the release of the drug to the patient.

Antioxidant Upregulators

In some embodiments, the antioxidant is also an antioxidant upregulator, which stimulates cells to produce their own endogenous antioxidants, such as catalase and superoxide dismutase (SOD).

Examples of antioxidants that are also antioxidant upregulators include resveratrol and pterostilbene. Pterostilbene is an antioxidant upregulator. Pterostilbene is a resveratrol analog naturally found in blueberries, which is chemically more stable than resveratrol.

Resveratrol may be included in the coating in an effective amount to protect the surrounding tissue, e.g. neural tissue, from an immune response to an implant, such as elicited by insertion or the prolonged presence of an electrode.

Pterostilbene may be included in the coating in an effective amount to protect the surrounding tissue from an immune response following implantation of the implant.

Recyclable Antioxidants

Recyclable antioxidants are generally antioxidants that are able to react with different types of reactive oxygen species (ROS), such that after reacting with one ROS, they are eventually able to return to their original oxidation state through natural interactions in the body, such as via reacting with one or more additional reactive oxygen species.

Cerium oxide nanoparticles may be incorporated, either chemically or electrostatically, into a polyelectrolyte component or the LbL assembled film.

Cerium oxide is a radical scavenger that changes from oxidation state from III to IV. Cerium (III) (reduced state) at the nanoparticle surface is able to react with reactive oxygen species and is oxidized to Cerium (IV) (oxidized state), and over time, Cerium (IV) reverts to its reduced state in a manner similar to a catalytic process. Thus, cerium oxide can be recycled in the body from oxidation state IV (cerium IV) to oxidation state III (cerium III), allowing for prolonged use of the agent following delivery. Antioxidants that are able to be reused following reaction with reactive oxygen species, in a similar manner, are referred to as recyclable antioxidants.

Methods for forming cerium oxide (ceria) nanoparticles (CNPs) are known. Some suitable methods for forming CNPs, particularly CNPs with diameters of 3-10 nm, are disclosed in Lee, et al., “High temperature decomposition of cerium precursors to form Ceria Nanocrystal libraries for Biological Applications”, Chem. Mater., 24, 424-432 (2012).

Thus, over time, as additional cerium oxide nanoparticles are released from the coating, and as at least a portion of the cerium oxide nanoparticles that were previously released are recycled, an greater amount of cerium oxide that is able to interact with ROS compared to the amount in a particular bilayer of the coating.

CNPs have been shown to be antioxidants due to oxygen vacancies in the crystal structure. These oxygen vacancies allow for recyclable radical scavenging through a cycle of reactions with different reactive oxygen species. Optionally, the CNPs are coated with a polymer with a hydrophilic tail, which allows the nanoparticles to stably suspend in aqueous solutions.

CNPs typically have a size in the range of 1-80 nm, 1-50 nm, 1-25 nm, 1-15 nm, 1-15, or 1-10 nm. Preferred CNPs have a dimension (e.g., diameter) of 10 nm or less, such as from 1-10 nm, 1-9 nm, 1-8 nm, 1-7 nm, 1-6 nm, or 3-10 nm, optionally the particles have a size of 5 nm or less, such as from 1-5 nm, 1-4 nm, 1-3 nm, 1-2 nm.

The CNPs are optionally coated with a hydrophilic molecule with a hydrophilic tail, such as a hydrophilic polymer, which allows the nanoparticles to stably suspend in aqueous solutions.

Suitable hydrophilic polymers for interacting with the surface of CNPs and forming a hydrophilic coating around the surface of the nanoparticle include but are not limited to polyalkylene oxides, such as polyethylene glycol (PEG); hydroxylated poly(methacrylates) such as poly(hydroxyethyl methacrylate) (PHEMA); hydroxylated poly(acrylates) such as poly(hydroxyethyl acrylate); neutral, hydrophilic polysaccharides, such as dextrin, cellulose, hydroxyethyl cellulose, amylose, and amylopectin; poly-L-serine, and poly(vinyl alcohol) (PVA) The hydrophilic polymer may be conjugated to an oxygen containing group to facilitate its interaction with the oxygen vacancies with cerium oxide, such as a nitro-substituted catechol, for example nitro L-dihydroxyphenylalanine (nitro-DOPA) and L-tyrosine.

The weight average molecular weight can vary for a given hydrophilic polymer but is generally from about 1000 Daltons to 500,000 Daltons, 1000 Daltons to 250,000 Daltons, 1000 Daltons to 100,000 Daltons, 1,000 Daltons to 10,000 Daltons, 1,000 Daltons to 25,000 Daltons, 3,000 Daltons to 20,000 Daltons, 3,000 Daltons to 15,000 Daltons, or 4,000 Daltons to 10,000 Daltons.

3. Linkage of Drug to Polymer

The drug can be conjugated to a polymer in one of the layers in the coating, such as via a hydrolytically degradable or enzymatically degradable linkage. Suitable linkages can be formed from dicarboxylic acids, succinic acid, glutaric acid, malonic acid, and triethylene glycol.

The linker may also include degradable linkages that are cleavable upon contact with an enzyme and/or through hydrolysis, such as ester, amide, anhydride, a thioester, and carbamate linkages. Typically, a linkage is between hydrophilic and hydrophobic parts of an amphiphilic molecule. In some embodiments, phosphate-based linkages can be cleaved by phosphatases. In some embodiments, labile linkages are redox cleavable and are cleaved upon reduction or oxidation (e.g., disulfide linkages —S—S—). In some embodiments, degradable linkages are susceptible to temperature, for example cleavable at high temperature, e.g., cleavable in the temperature range of 37-100° C., 40-100° C., 45-100° C., 50-100° C., 60-100° C., 70-100° C. In some embodiments, degradable linkages can be cleaved at physiological temperatures (e.g., from 36 to 40° C., about 36° C., about 37° C., about 38° C., about 39° C., about 40° C.). For example, linkages can be cleaved by an increase in temperature. This can allow use of lower dosages, because agents are only released at the required site. Another benefit is lowering of toxicity to other organs and tissues. Stimuli to induce release of drug from the polymer can be ultrasound, temperature, pH, metal ions, light, electrical stimuli, electromagnetic stimuli, and combinations thereof.

Typically, when the drug is conjugated to the polymer it is in its inactive form and is also referred to herein as a prodrug.

4. Other Therapeutic, Prophylactic, and Diagnostic Agents to Encapsulate in the Coating/Film

Optionally, in addition to the antioxidants described above, the coating may containing another active agent.

A wide range of active agents may be included in the coatings. These may be proteins or peptides, sugars or carbohydrate, nucleic acids or oligonucleotides, lipids, small molecules, or combinations thereof. In some embodiments, the coatings have the active agents encapsulated therein, dispersed therein, and/or covalently or non-covalently associated with one or more layers in the coatings.

Optionally, the additional active agent is a second antioxidant. Antioxidants that can be included in the coating as the additional active agent include vitamin C, carotenoids, polyphenols (e.g., flavonoids such as apigenin, luteolin, tangeritin, isohametin, kaempferol, myricetin, proanthocyanidins, quercetin, eriodictyol, hesperetin, naringenin, catechin, gallocatechin, epicatechin, and derivatives thereof; phenolic acids and their esters such as chicoric acid, chlorogenic acid, cinnamic acid, ellagic acid, ellagitannins, gallic acid, gallotannins, rosmarinic acid, or salicyclic acid; and other nonflavonoid phenolics such as curcumin, flavonolignans, xanthones, or eugenol), and vitamin cofactors and minerals.

Exemplary classes of therapeutic agents include, but are not limited to, analgesics, anti-inflammatory drugs, anti-proliferatives such as anti-cancer agent, anti-infectious agents such as antibacterial agents and antifungal agents (e.g., levofloxacin, CIPRO, ciprofloxacin, cephalexin, ZOTRIM, BACTRIM, MACROBID, nitrofurantoin, fosfomycin, methenamine hippurate, TRIMPEX, PROLOPRIM, trimethroprim, nalidixic acid, and phenazopyridine), antihistamines, corticosteroids, dopaminergics, anticoagulants (e.g., heparin and others to treat ischemic stroke), and muscle relaxants. Other classes of therapeutic agents include those that promote regeneration of tissue including growth factors.

Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Examples of other suitable contrast agents include gases or gas emitting compounds, which are radiopaque. Nanoparticles can further include agents useful for determining the location of administered particles. Agents useful for this purpose include fluorescent tags, radionuclides and contrast agents.

These agents can also be used prophylactically.

5. Features of Coating

Drug release generally takes place following disassembly of the outer layer(s) of the film at physiological environments (e.g., with a body fluid salt concentration such as phosphate-buffered saline) at body temperature. An inducible rapid disassembly via electrical stimulation may also be applied for an initial bulk release of drug treatments (e.g., neuroprotective agents) at the time of implantation.

Generally, the coating has a suitable structure to prevent leaching of the antioxidant or prodrug thereof prior to disassembly of the layer containing the agent. This provides for greater control over the release rate of the antioxidant.

The coating may release different amounts of the antioxidant(s) or other drugs incorporated therein over time. For example, the coating can have different concentrations of an antioxidant in different layers, resulting in the release of different amounts of the antioxidant over time following implantation in a patient.

The coating may contain multiple different drugs and can provide controlled release of the different drugs, as well. For example, a first drug or prodrug can be conjugated to one layer and a second, different drug or prodrug can be conjugated to or incorporated in a different layer in the coating. The first drug or prodrug can be released when its layer disassembles, while the second drug or prodrug remains in the coating, until a later time, when its layer disassembles.

The coating can release one or more of the drugs or prodrugs therein over a period of at least about 14 months, 1 year, 10 months, 8 months, or 6 months following implantation.

Micelles, liposomes or other colloidal carriers and nanomaterials such as nanoparticles may also be used as drug carriers that can be directly introduced into multilayer thin films as a means to further control release of active agents from the surface of devices.

6. Thickness

The coating has a suitable thickness to provide the desired release profile for the drugs and prodrugs included therein. Typically the coating is at least 10 nm thick, optionally the coating is at least 1 micron thick. The coating can have a thickness of up to about 10 microns. Suitable ranges for the coating thickness include 10-20 nm, 20-30 nm, 30-40 nm, 40-50 nm, 50-100 nm, 100-200 nm, 100-500 nm, 500-1000 nm, 1-10 microns, 2-10 microns, 3-10 microns, 4-10 microns, 5-8 microns, 6-10 microns, 7-10 microns, 8-10 microns, and 9-10 microns. Suitable thicknesses include at least 10 nm, e.g., 10-100 nm, 100-500 nm, 100-1000 nm, or 1 nm-10 microns, 100 nm-10 microns, 500 nm-10 microns. Suitable thicknesses also include at least 1 μm, e.g., 1, 2, 3, 4, 5, 6, 7, 8, 9, or 10 μm thick.

7. Multiple Polymeric Layers

The coating typically includes more than one layer of polymers. In electrostatically associated polymer layers, the film may include a positively or negatively charged polymer as a first layer, depending on the surface property of the substrate, and a second layer of polymer that is oppositely charged compared to the first layer, optionally followed by a third layer of polymer that is oppositely charged compared to the second layer (e.g., same charge as to the first layer; but may be of different composition compared to the first layer), etc. Bilayers generally include two layers of polymers of opposite charges.

An antioxidant may be conjugated to or associated with one or more layers of polymer or bilayers in the film. Optionally, an additional active agent is included in one or more layers or bilayers in the film. With electrostatically assembled coatings, one or more bilayers are present in the coating, for example, about 10 bilayers, 20 bilayers or even 60 bilayers.

In some embodiments, the coating contains a plurality of layers of drug-polymer conjugates and layers containing polymers lacking drugs or prodrugs.

In some embodiments, the coating includes one or more bilayers that do not contain an active agent and one or more bilayers that contain an active agent. For example, the coating may contain a first bilayer with an active agent incorporated therein alternating with a second bilayer that does not contain an active agent therein. This design can facilitate control of the release of the active agent over time.

II. Devices with a Coating on an Outer Surface

Any medical device that is partially or wholly introduced, inserted, or implanted within a subject's body may be coated on an outer surface with a coating described herein. The medical device may be partially or wholly inside the patient. If a medical implant is only partially inside a patient, then it contains internal and external parts, relative to the patient. In these embodiments, typically only the outer surfaces that are internal relative to the patient may be covered with the coating, as these surfaces come in contact with the patient's internal tissues.

The coating conforms to the surface to which it is applied.

The device can have any suitable material on the outer surface for attaching the coating. Typically the material on the outer surface of the device to be coated is biocompatible. For devices to be implanted for long periods of time, the material is preferably inert in the presence of biological fluids and at body temperature.

Examples of medical implant that are typically wholly embedded in a subject, include but are not limited to prosthetic joints, a prosthetic heart valves, cardiovascular stents, neural implants, visual prosthetics, retinal implants, some dental implants, and pacemakers. Examples of medical implants that are typically only partially embedded in a patient include but are not limited to urinary catheters, gastric feeding tubes, and some a dental implants.

Some medical devices that include at least one outer surface with a coating are removable without intervention or aide by a medical professional, such as for example, a mouth guard, removable dentures, an orthodontic retainer, or a contact lens.

The coating may be on the surface of a medical device that remains in a patient's body for prolonged time periods, such as at least 3 months, at least 6 months, at least one year, at least 1.5 years, at least 2 years, or even longer time periods, such as for up to 5 years or up to 10 years.

Orthopedic Implants

The medical implant that contains a coating on one or more (optionally all) of its outer surfaces can be an orthopedic implant. An orthopedic implant generally replaces anatomy or restores a function of the musculoskeletal system such as the femoral hip joint; the femoral head; acetabular cup; elbow including stems, wedges, articular inserts; knee, including the femoral and tibial components, stem, wedges, articular inserts or patellar components; shoulders including stem and head; wrist; ankles; hand; fingers; toes; vertebrae; spinal discs; artificial joints; and orthopedic fixation devices such as nails, screws, staples, and plates.

Dental Implants

The medical implant that contains a coating on one or more (optionally all) of its outer surfaces can be a dental implant. A dental can be implanted into the oral cavity of a vertebrate animal, in particular a mammal such as a human, in tooth restoration procedures. For instance, a dental implant typically includes a dental fixture (or post) coupled to secondary implant parts, such as an abutment and/or a dental restoration such as a crown, bridge or denture.

Neural Implants

The medical implant that contains a coating on one or more (optionally all) of its outer surfaces can be a neural implant. A neural implant typically includes one or more electrodes that can be placed in contact with neuronal tissue in an animal host and can record and/or stimulate neural signals from or to the neuronal tissue. Neural probes typically include electrically conductive and electrically non-conductive surfaces designed for contact with neuronal tissue when implanted in a subject, and can include one or more electrodes that can be independently monitored from other electrically conductive surfaces for recording and/or stimulating neural signals.

The neural implant can be used for chronic recording and/or stimulation of neural signals from a subject. For example a neural implant with the coating described herein can be implanted into neuronal tissue of the subject, and used to record and/or stimulate neural signals from the subject for a period of at least 6 months (such as at least 12, 18, 24, 30 or 36 or more months) without deterioration of quality or quantity of the recorded or stimulated neural signal.

The medical implant may contain a plurality of probes, such as an array or a deep brain stimulator, for recording and/or stimulating a neural signal in a subject. Methods of making electrodes for recording and/or stimulating a neural signal that are typically fully or partially coated with an insulting layer (such as a parylene C insulating layer).

Exemplary neural implants that contains a coating on one or more (optionally all) of its outer surfaces that are in contact with neural tissue include, but are not limited to neurostimulators (such as spinal cord stimulator), cochlear implants, and epilepsy closed loop stimulators.

Any neural implant for recording and/or stimulating neural signals in a subject may be used with the disclosed embodiments. In several embodiments, the neural implant includes more than one electrode, such as an array of electrodes. In additional embodiments, a device is provided that can include one or more probes, each of which can include one or more electrodes. Non-limiting examples include deep brain stimulators, EcoG grids, electrode arrays, microarrays (e.g., Utah and Michigan microarrays), and microwire electrodes and arrays. Neural implants (and devices including them) can be inserted into the body, for example transcutaneously, intervertebally, or transcranially, to a target site in the body (for example, in the brain) where neural signals are to be recorded or stimulated. Commercial sources of neural implants and devices for recording and/or stimulating neural signals in a subject, including implants coated with an insulating layer (such as Parylene C), are known. For example, such electrodes and devices are available commercially from Blackrock Microsystems (Salt Lake City, Utah) and NeuroNexus (Ann Arbor, Mich.).

Additional Devices

Additional devices that may be coated with the coatings described herein include, but are not limited to, microelectrodes, light-based therapies, a variety of prosthetics, such as retinal prosthetics, cardiovascular stents, and pacemakers.

Effective Amount of Antioxidant or Antioxidant Upregulator in Coating

The effective amount of the antioxidant (or antioxidants) in the coating and the thickness of the coating that is on the surface of the implant depends on the particular application. In some embodiments, an effective amount of the antioxidant (or antioxidants) in the coating is an amount sufficient to reduce deleterious effects, such as biofilm formation or fouling, following implantation of the medical implant over time (for example over 3 months, over six months or over one year) compared to implantation of the same implant without the coating. For example, for neural implants, an effective amount of the antioxidant (or antioxidants) in the coating can be an amount sufficient to reduce deleterious effects on neural recording quality over time (for example over 6 months or over 1 year) compared to the same electrode that does not contain the coating.

In some embodiments, an effective amount of amount of the antioxidant (or antioxidants) in the coating is sufficient to allow recording from at least one electrode after the probe has been implanted for at least six months, optionally after implantation for at least one year, optionally for longer than one year, such as for up to two years.

### III. Methods of Making

Multilayer build-up is generally supported by one or more attractive forces acting cooperatively, typical for high-molecular weight building blocks, while electrostatic repulsion provides self-limitation of the absorption of individual layers. The multilayering assembly and wash steps can be performed in many different ways including dip coating, spin-coating, spray-coating, flow based techniques and electro-magnetic techniques.

FIG. 2 depicts an exemplary method for coating a surface of a device or a portion of a device. In FIG. 2 the device is an electrode. A layer-by-layer (LbL) film may be formed to coat the surface or a portion of the surface of an electrode (e.g., made with glass or parylene). Although an electrode is illustrated, other devices with different geometries can be coated in the same manner

Forming One or More Base Layers

The device can have any suitable material on the outer surface for attaching the coating. However, if the surface of the device is not conducive for attachment of the coating, then one or more, typically a plurality of base layers may be deposited prior to forming the coating. The base layer may be used to increase the presence of negative or positive charges on the surface. The base layer maybe formed via plasma etching the surface of the electrode (or other device) to make it negatively charged, followed by successively depositing (or submerging the device or surface to be coated in) linear poly(ethylene immine) (LPEI; cationic polymer) and poly(sodium 4-styrenesulfonate) (SPS; anionic polymer).

Generally, silicon glass and parylene do not require the application of a base layer prior to attachment of the layers in the coating.

Forming the Coating

A layer by layer (LbL) film can be prepared via alternating adsorption of complementary multivalent species on a substrate via electrostatic interactions, hydrogen bonding, or other secondary interactions. In some embodiments, the LbL coating is prepared from polymers that interact with each other via electrostatic interactions. A film can be prepared by a dipping method in which the device to be coated, such as an electrode, is submerged in a solution of cationic polymer and subsequently submerging the electrode in a solution of anionic polymer. These steps are repeated to form multiple layers in the coating. Optionally between submersion steps in the different polymer solutions, the device is washed with water or a wash solution, such as phosphate-buffered saline (PBS), one or more times. During the wash step, excess polymers that are not adsorbed on to the substrate are removed prior to submersion in the next polymer solution.

Optionally, drug is incorporated in one of the polymer solutions, for example the drug may be chemically conjugated to one of the polymers, or the drug may be mixed into the polymer solution.

The device may be held by an automated dipper arm and submerged into each polymer solution. Depending on the geometry and desired coverage of the device, the device can be placed in a mesh strainer and submerged into each polymer solution.

The coating is generally a multilayer film, which contains at least a first layer and a second layer adjacent to the first layer wherein the first layer comprises a first polymeric material and at least first moiety wherein the second layer comprises a second polymeric material and at least second moiety, where the charge on the second moiety is opposite the charge on the first moiety or wherein the first and second moieties otherwise have affinity for each other. The first and second moieties on the adjacent layers interact with one another so that the adjacent layers associate with each other into a film (e.g. via non-covalent interactions, such as via electrostatic interactions or hydrogen bonding). Additionally, the drug for delivery (an antioxidant, optionally a combination of, optionally with another drug) is incorporated into at least one layer or between layers such that decomposition of one or more layers of the coating results in release of the antioxidant and/or other active agent.

Suitable layer by layer methods for forming films or coatings on devices are also described for example in U.S. Pat. No. 8,234,998, U.S. Publication No. 2009/0088679 to Wood, et al., U.S. Publication No. 2012/0277719 to Shukla, et al., U.S. Publication No. 2014/0186724 to Hammond, et al., and U.S. Publication No. 2015/0250739 to Demuth, et al.

### IV. Methods of Using

A layer by layer film as a coating on devices provides nanometer level control of the composition of a thin film, and the generation of highly complex, tailor-made coating compositions. Thin films may be constructed in water at room temperature, preserving the activity of sensitive small molecules, and other active agents.

A layer by layer coated device may enhance longevity of useful life of the device in vivo, and/or provide controlled release of therapeutics from device surfaces to modulate tissue responses and/or device performance.

The coating on the device is able to release one or more antioxidants, preferably a recyclable antioxidant or antioxidant that is also an antioxidant upregulator or a combination thereof, optionally with another drug, in a controlled manner for an extended time period. Generally, only the outermost layer of the coating releases the drug incorporated therein at a time.

Following implantation, the devices containing the coating described herein may have a reduced biofilm formation, reduced biofouling on the surface of the device and/or reduced corrosion of a metal surface on the device, compared to the same device in the absence of the coating.

Following implantation, the devices containing the coating described herein may be useful in vivo for at least 6 months longer than the same device without the coating.

For coatings containing pterostilbene therein, the coating may release an effective amount of pterostilbene to achieve a stable therapeutic concentration of pterostilbene in the tissue surrounding the implanted device. Generally a therapeutic concentration for pterostilbene in the surrounding tissue is less than 400 μM, and can be in the range of 10 to 200 μM, 10 to 100 μM, or 10 to 50 μM, optionally the concentration of pterostilbene is approximately 25 μM.

For coatings containing cerium oxide nanoparticles, the coating may release an increasing dosage of the CNP over time. This can be useful in case the foreign body response increases production of ROS overtime.

An exemplary use of a coated electrode includes insertion of it into brain at a site of interest. The electrode has a film of alternating layers of a cationic polymer and an anionic polymer on the side, away from the contact tip. The layers of polymer may disassemble, generally from the exterior layer, following electrode implantation. FIG. 3 depicts an exemplary layer of polymer that includes an antioxidant conjugated to a poly-L-lysine (PLL) via a succinic acid linkage. Hydrolysis in vivo may degrade the linkage between the antioxidant and the rest of the polymer, leaving behind PLL-succinic acid, a biocompatible portion, and releasing an active antioxidant that may neutralize reactive oxygen species (ROS). This long term local treatment of the tissue interface with antioxidants can disrupt the inflammatory signalling cascade and provide a viable defense against long term oxidative damage and neurodegeneration. An increased functional recording life of implanted neural recording electrode arrays has the potential to increase the long term quality of life for patients living with paraplegia.

Another exemplary use of a coated device for implantation is to inhibit growth of pathogens and prevent formation of bio-films. Charged multivalent species of a polymer may in itself be antimicrobial. Conjugated or otherwise associated agents may be antimicrobial agents.

The present invention will be further understood by reference to the following non-limiting examples.

### EXAMPLES

Materials and Methods

Synthesis of Prodrugs

Two prodrugs, of two antioxidants, resveratrol and pterostilbene, were synthesized.

Resveratrol-SA and Pterostilbene-SA were both formed by ring opening synthesize with succinic anhydride. Drug was added to succinic anhydride in THF with tributyl amine and allowed to react overnight. The THF was evaporated off and the white powder was washed with Ethyl Acetate and H₂O (pH4) in a separation funnel. The drug was collected from the ethyl acetate, dried and rotovaped, to form a white powder. The Drug-SA conjugate is an intermediate conjugate that can be attached to a charged polymer.

For example, each of Resveratrol-SA and Pterostilbene-SA can be conjugated separately to a cationic polymer, such as polylysine via its amine groups. Following a purification process, NMR long range coupling analysis can be conducted of the purified sample to characterize the final prodrug-polymer conjugate.

Synthesis of Cerium Oxide Nanoparticles with Hydrophilic Tail

15-20 mg (Nitro-DOPA)-PEG (5KD) was dissolved in 1 ml chloroform. Cerium oxide nanoparticles were suspended in diethylether (10 mg/ml). 1 ml Nitro-DOPA-PEG solution and 1 ml Ceria colloidal solution were mixed at room temperature for 10 min 5 ml DI water was added to the system. The mixture was stirred for over 24 hours to allow the organic solvent evaporate. The mixture was purified by filtering with a 0.2 micron syringe filter.

Formation of LbL Films

Layer by layer (LbL) films were prepared by a dipping method in which the material to be coated was submerged in polymer solutions, alternating between cationic and anionic polymer solutions. In some embodiments, base layers were deposited before dipping in polyelectrolyte solutions: via plasma etching to deposit linear poly(ethylene immine) (LPEI) and poly(sodium 4-styrenesulfonate) (SPS).

Successful growth of the film was assessed through testing polymer-polymer interactions with a Quartz Crystal Microbalance and thickness with a profilometer and scanning electron microscope.

The thickness for each film was analyzed via transmission electron microscopy (TEM), scanning electron microscopy (SEM), and/or profilometry.

General Method:

LbL dipping was performed at room temperature using an automated robotic arm. unless otherwise specified. A suitable robotic arm is a Microm DS-50 slide stainer.

Substrates were washed with methanol three times and DI water three times before being plasma etched for 1 minute. Samples were immediately submerged in a cationic polymer solution.

If the method includes the formation of base layers, the sample is submerged into a 10 mM (pH 4.25) solution of linear polyethyleniemine (LPEI, 20,000 MW, Polysciences Inc. cat#23966-1).

For methods without base layers, samples were submerged directly into a 2 mg/mL solution of Poly-L-lysine (PLL, 30,000-70,000 MW, Sigma cat# P2636) in 1×PBS.

Samples were then loaded into the holder of the dipper arm.

Formation of Base Layers

The formation of base layers was performed by dipping the substrate into alternating solutions of 10 mM (pH 4.25) LPEI and 10 mM (pH4.75) poly(sodium 4-styrenesulfonate) (SPS, 70,000 MW, Sigma cat#243051) using the following method:

1. LPEI for 5:00 minutes
2. H₂O for 0:10 minutes (i.e. 10 seconds)
3. H₂O for 0:20 minutes (i.e. 20 seconds)
4. H₂O for 0:30 minutes (i.e. 30 seconds)
5. SPS for 5:00 minutes
6. H₂O for 0:10 minutes (i.e. 10 seconds)
7. H₂O for 0:20 minutes (i.e. 20 seconds)
8. H₂O for 0:30 minutes (i.e. 30 seconds)
9. Repeat steps 1-8 nine more times to form 10 complete bilayers.

Formation of LbL Films

The formation of the LbL film, unless otherwise specified was performed with the same robotic dipping system, using alternating solutions of 2 mg/mL PLL in 1×PBS and 2 mg/mL poly-L-glutamic acid (PGA, 50,000-100,000 MW, Sigma cat# P4886) in 1×PBS using the following method:

1. PLL for 15:00 minutes
2. lx PBS for 5:00 minutes
3. PGA for 15:00 minutes
4. lx PBS for 5:00 minutes
5. Repeat steps 1-4 thirty-nine more times to form 40 complete bilayers.

### Example 1: Formation of LbL Films, Optionally with Base Layers, without Drug in any Layers on Flat Glass and Parylene Substrates

The general method described above was used to form four different films on flat substrates (1 cm×3.5 cm). Each test was repeated 3 times:

1. PLL/PGA without drug in any of the layers (40 bilayers) with 10 LPEI/SPS base layers on flat silicon glass substrate
2. PLL/PGA without drug in any of the layers (40 bilayers) with no base layers on flat silicon glass substrate
3. PLL/PGA without drug in any of the layers (40 bilayers) with 10 LPEI/SPS base layers on flat parylene substrate
4. PLL/PGA without drug in any of the layers (40 bilayers) with no base layers on flat parylene substrate.

### Example 2: Formation of LbL Films, Optionally with Base Layers, without Drug in any Layers on Single Shank Electrodes

The general method described above was used to form four different films on single shank electrodes from Alpha Omega. The electrodes used were Tungsten in Example 2 were coated in glass, and Platinum/iridium coated in parylene. Each test was performed one time:

1. PLL/PGA without drug in any of the layers (40 bilayers) with 10 LPEI/SPS base layers on glass coated electrode
2. PLL/PGA without drug in any of the layers (40 bilayers) with no base layers on glass coated electrode
3. PLL/PGA without drug in any of the layers (40 bilayers) with 10 LPEI/SPS base layers on parylene coated electrode
4. PLL/PGA without drug in any of the layers (40 bilayers) with no base layers on parylene coated electrode

### Example 3: Formation of LbL Films with Base Layers, without Drug in any Layers on Spherical Implants

Example 3 used the pretreatment (washes, plasma etching) and the concentrations of polymers and timing of each dip as described in the general method above, however the dipping process was performed by placing the beads in Ependorph tubes and adding and removing the polymer liquids and water/PBS washes by hand (i.e. without the robotic arm).

Approximately 20 beads were coated in each Ependorpf (1.5 ml) tube. The beads had two sizes, 100 μm and 200 μm diameter beads. This test was performed one time.

For Example 3, 10 bilayers (instead of 40 bilayers) and 5 base layers (instead of 10 base layers) were formed.

1. PLL/PGA without drug in any of the layers (10 bilayers) with 5 LPEI/SPS base layers on glass beads

### Example 4: Formation of LbL Films with Base Layers with Drug in Bilayers on Flat Glass and Parylene Substrates

The general method described above was used to try to form three different films on flat substrates (1 cm×3.5 cm) with an antioxidant or an antioxidant upregulator incorporated therein. The general method described above was modified to include a drug in one of the solutions as indicated in each set up described below.

Test 1 was repeated twice. Tests 2 and 3 were performed one time.

1. PLL/PGA with CNPs with hydrophilic tails as described above (diameters in the range of 4.9 nm to 9.3 nm) in the PLL solution at 140 ppm (40 bilayers) with 10 LPEI/SPS base layers on flat silicon glass substrate (successful film growth)
2. PLL/PGA with CNPs with hydrophilic tails as described above (diameters in the range of 4.9 nm to 9.3 nm) in the PGA solution at 140 ppm. However the CNPs crashed out of solution (unsuccessful at forming film)
3. PLL/PGA with 200 uM pterostilbene stabilized by 2% DMSO in the PGA solution and theoretically trapping it between the layers (40 bilayers) with 10 LPEI/SPS base layers on flat silicon glass substrate (successful film growth)

Results

Initial chemical synthesis yielded successful formation of both intermediate conjugates, resveratrol-succinic acid and pterostilbene-succinic acid. Both intermediate conjugates, i.e., succinic acid modified antioxidants, were characterized and confirmed by mass spectroscopy, as well as Fourier transform infrared spectroscopy. Characterization showed the presence of the expected molecular weights of each synthesis product a new FTIR peak corresponding to a newly emerged carbonyl group in each product. Successful conjugation of pterostilbene and resveratrol to a succinic acid intermediate molecule was confirmed by the presence of specific molecular weight in mass spectroscopy and a newly emerged peak in Fourier transform infrared spectroscopy

Several successful LbL films containing poly-L-glutamic acid (PGA) and poly-L-lysine (PLL), optionally with base layers, were formed on silicon and parylene surfaces of different geometries, including flat, spherical, and cylindrical (electrode).

In order to make the drug delivery coating translatable to different designs of neural recording devices, the film was assayed for its ability to grow on two major electrode insulation materials, glass (silicon) and parylene. Initial film growth was tested on both substrates and profilometry characterization, and the result showed a film thickness from 40 bilayers of polymers forming a film thickness of 4-5 microns on both parylene C and glass/silicon coatings (5.11 μm on silicon wafers and 4.47 μm on parylene coated layers). While statistical analysis of these measurements indicates that the film on parylene is significantly thinner than that on silicon, this difference might be easily overcome by the addition of more polymer bilayers.

The coating on the spherical glass beads was examined using focused ion beam scanning electron microscopy. A layer of platinum was deposited on the surface to protect the thin film and then a gallium ion beam was used to ablate the surface of the bead to view its cross-section. It was found that with 10 bilayers of the film, a 200-400 nm film was formed on the surface of the bead.

The presence of CNPs encapsulated in coatings formed from CNPs mixed in the PLL solution was confirmed via TEM by depositing two bilayers of CNP loaded film on a TEM grid. Increased fluorescence was seen at 480 nm for the PLL+CNPs/PGA films.

For release study of drug from these films, at predetermined time points, a sample of the PBS incubating the film may be collected, and the concentration of drug may be analyzed by quantitative fluorescence via plate reader based on unique fluorescences of both parylene and resveratrol. An additional release may be performed with PBS in the presence of enzymes to determine a more physiologically relevant release. Parameters of the LbL preparation, such as number of bilayers and drug solution concentration, may be altered to produce desired drug release and concentration profiles. If a released drug from the film/coating is able to reduce oxidative stress measurements in vitro to the same levels as that of the pure drug, it is generally considered bioactive after release.

Because electrodes require an exposed contact in order to record from the tissue, exposure of this contact after LbL deposition can be analyzed by measuring impedance before and after film deposition, as impedance of the electrode would increase if the contact was covered in a film. Under the condition that the film deposition includes growth over the contact, a process of rapid film disassembly may be conducted at the contact exposure through electrical stimulation. Under electrical stimulation, the electrostatic bonds holding the film together can be broken, and the layers forced to disassemble. The inducible rapid disassembly may also be a viable method for an initial bulk release of neuroprotective drug treatments at the time of implantation.

In additional studies, results showed pterostilbene and resveratrol were comparable in treatment of oxidative stress in an oxidative stress in vitro model.

A preliminary layer-by-layer study showed that growth of PGA/PLL films were compatible (based on visual inspection and when imaged via SEM) with glass and parylene coated electrodes.

These studies were believe to show that a long term release of resveratrol, or resveratrol-like drug, may prevent oxidative damage and have a long term neuroprotective capacity: providing a successful suppression of the neuroinflammatory response and long term foreign body response to chronic neural implants for long term study and treatment of neural disease states.

For an in vivo study, a porcine model may be used for the implantation of the prodrug-polymer film coated recording device due to the similarities in cranial anatomy, inflammatory response, and ability for the same animal model to be used in future translation studies beyond the fundamental platform technology development.

For further in vitro testing, assays may be performed on primary brain cells dissociated from E18 rats, providing both neurons and glial cells (astrocytes and microglia) that are essential to the neuroinflammatory response. Oxidative stress in primary brain cells (including neurons, astrocytes, and microglia) may be induced through hyperoxic culture conditions, and then assayed with increasing concentrations of the pure antioxidant drugs, resveratrol and pterostilbene. The degree of oxidative stress may be measured by ROS assay, lipid peroxidation assay, and live/dead assay. One preferred concentration of antioxidant is one with a largest decrease in oxidative stress compared to untreated controls.

The material/chemical may be assayed for neurotoxicity. Neural cells may be fed with large concentrations of each material/chemical dissolved in cell media. A material/chemical showing toxicity at a high concentration may then be tested for toxicity at varying dilutions in order to determine the toxic concentration. Toxicity can be measured by a live/dead assay with qualitative fluorescence by microscope and quantitative fluorescence by plate reader.

Publications cited herein and the materials for which they are cited are specifically incorporated by reference.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other embodiments the values may range in value either above or below the stated value in a range of approximately +/−5%; in other embodiments the values may range in value either above or below the stated value in a range of approximately +/−2%; in other embodiments the values may range in value either above or below the stated value in a range of approximately +/−1%. The preceding ranges are intended to be made clear by context, and no further limitation is implied.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Those skilled in the art will recognize, or be able to ascertain using no more than routine experimentation, many equivalents to the specific embodiments of the invention described herein. Such equivalents are intended to be encompassed by the following claims.

We claim:

1. A coating comprising a first bilayer comprising a first layer comprising a first polymer comprising cationic functional groups and a second layer comprising a second polymer comprising anionic functional groups, wherein the bilayer further comprises a drug, wherein the drug is an antioxidant.

2. The coating of claim 1, wherein the antioxidant is an antioxidant upregulator or a recyclable antioxidant.

3. The coating of claim 1, wherein the drug is conjugated to at least one of the polymers and is in the form of a prodrug.

4. The coating of claim 2, wherein the polymer comprising cationic functional groups is conjugated to the drug forming the prodrug.

5. The coating of claim 2, wherein the polymer comprising anionic functional groups is conjugated to the drug forming the prodrug.

6. The coating of claim 1, further comprising a second bilayer, wherein the second bilayer does not contain the drug or any other drug.

7. The coating of claim 1, wherein the coating comprises at least 10 bilayers, at least 20 bilayers, at least 40 bilayers, at least 50 bilayers, or more.

8. The coating of claim 1, wherein the coating is at least 10 nanometers thick, optionally wherein the thickness of the coating is in the range of 10 nm to 10 microns.

9. The coating of claim 1, further comprising a second drug, wherein the second drug is a second antioxidant that is different than the first drug.

10. The coating of claim 1, wherein the drug is selected from the group consisting of pterostilbene, resveratrol, and cerium oxide nanoparticles.

11. The coating of claim 9, wherein the first drug is pterostilbene or resveratrol, and wherein the first drug is conjugated one of the polymers, and wherein the second drug is cerium oxide nanoparticles.

12. The coating of claim 11, wherein the first and second drugs are in the same bilayer.

13. The coating of claim 11, wherein the coating comprises multiple bilayers and wherein the first and second drugs are in different bilayers.

14. The coating of claim 1, wherein the coating comprises coating comprises multiple bilayers, wherein the bilayers are aligned such that a second bilayer alternates with the first bilayer, and wherein the second bilayer does not contain the drug.

15. The coating of claim 1 on the surface of an implantable device.

16. An implantable device comprising the coating of claim 1.

17. The device of claim 16, further comprising a base coating between an outer surface of the device and the coating.

18. The device of claim 17, wherein the base coating does not contain the drug, optionally wherein the base coating contains a different polymer than the polymers in the bilayer.

19. The device of claim 16, wherein the device comprises an electrode.

20. The device of claim 16, wherein the device is selected from the group consisting of a dental implant, a neural tissue implant, an orthopedic implant, a cochlear implant, visual prosthetic implant, cardiovascular implant, such as a cardiovascular stent or a pacemaker.
